(19) United States
(12) Patent Application Publication
(10) Pub. No.: US 2006/0052667 A1
(43) Pub. Date: Mar. 9, 2006

(54) System and method for in vivo detection of h. pylori

(76) Inventors: Yoram Palti, Haifa (IL); Shlomo Lewkowicz, Kiryat Tivon (IL); Yoram Ashery, Rehovot (IL)

Correspondence Address: PEARL COHEN ZEDEK, LLP, 10 ROCKEFELLER PLAZA, SUITE 1001, NEW YORK, NY, 10020, US

(21) Appl. No.: 10/532,808

(22) PCT Filed: Oct. 30, 2003
(86) PCT No.: PCT/IL03/00893

§ 371 Date: Apr. 26, 2005

Related U.S. Application Data
(60) Provisional application No. 60/422,483, filed on Oct. 31, 2002.

(51) Int. Cl. A61B 1/06

(52) U.S. Cl. 600/160; 600/118

(57) ABSTRACT

A method and system for in vivo detection of H. pylori. The method according to one embodiment includes sensing pH in at least one location proximate to a patient's stomach mucus and transmitting by radio frequency pH data to an external receiving unit. The pH data may be part of an in vivo image.

### FIELD OF THE INVENTION

The present invention relates to the field of in vivo diagnosis. More specifically, the present invention relates to in vivo detection of H. pylori.

### BACKGROUND OF THE INVENTION

Heliobacter pylori (H. pylori) is a bacterium residing in the mucus lining of the stomach and duodenum. Bacteria can be found in the stomach of about half of the world population. H. pylori is believed to be the cause of gastritis, which is the underlying condition that causes ulcers and other digestive tract diseases, including cancer.

The stomach environment is highly acidic and contains digestive enzymes. H. pylori is able to survive the severe conditions in the stomach by taking shelter in the mucus layer, which is intended to protect the stomach lining, and by creating a local micro-environment of strong bases which can neutralize the acidic gastric juices in the vicinity of the bacteria. The bacterium utilizes its urease enzyme to produce strong bases by converting urea that is found in large quantities in the stomach, into bicarbonate and ammonia, both of which may be strong bases.

It is believed that the immune response to H. pylori, namely, inflammation of the stomach lining, may be the eventual cause of gastritis. There is strong evidence that H. pylori increases the risk of gastric cancer and that eradication of the bacteria prevents relapses after resection of early gastric cancer. Furthermore, infected patients going through eradicating therapy early in follow up, do not develop cancer, as opposed to untreated infected patients. Thus, detection, particularly early detection of the bacteria may prove to be important in management of H. pylori infection.

Several diagnostic kits exist for the detection of H. pylori in the upper gastrointestinal (GI) tract. These include breath tests, blood tests and endoscopy. Breath tests utilize solutions of urea containing isotopic carbon that are drunk by the patient prior to the breath test. If H. pylori is present the urea will be broken down and isotopic carbon will be detectable in the patient's breath. Blood tests check for the presence of antibodies to H. pylori in a patient's blood. It is noted that antibody levels in the blood may remain high even after bacteria are no longer present in the stomach. Endoscopy usually includes taking a biopsy for later in vitro testing. U.S. Pat. No. 6,228,605 to Marshall and U.S. patent application Ser. No. 09/824870, published under Publication Number 20010012623, to Marshall, describe a method for detecting H. pylori in the stomach by utilizing an endoscope and dense carriers for causing urea and pH sensitive color reagents to migrate to the mucus. If H. pylori is present, ammonia will be produced at the mucus and the pH sensitive reagents will react to the basic surrounding by going through a color change, which can be visualized by an endoscope.

### SUMMARY OF THE INVENTION

Embodiments of the present invention may utilize inherent H. pylori urease activity for the detection of the bacteria.

A method, according to one embodiment of the invention, includes contacting an in vivo sensing device with the stomach wall (mucus). Endo-luminal pH in the vicinity of the mucus or any products of the urease enzymatic reaction products (e.g. ammonia and/or bicarbonate) can thus be detected by the in vivo sensing device, thereby enabling in vivo and typically in situ detection of H. pylori. According to one embodiment the in vivo sensing device is an autonomous, self contained wireless sensing device capable of transmitting in vivo data to an external receiving unit A method according to another embodiment may include ingesting urea and inserting into the upper GI tract an in vivo sensing device. Endo-luminal pH or other enzymatic reaction products can be detected by the in vivo sensing device, thereby enabling in vivo detection of H. pylori. According to one embodiment the in vivo sensing device is an ingestible wireless device, which includes an appropriate sensor (such as a pH sensor) and which can transmit data (e.g. data regarding pH) to an external receiving unit, such as an ambulatory recorder. In alternate embodiments the device may be wired to an external unit that receives data from the device. According to some embodiments the in vivo sensing device includes an imaging unit.

### BRIEF DESCRIPTION OF THE DRAWINGS

The present invention will be understood and appreciated more fully from the following detailed description taken in conjunction with the appended drawings in which:

FIG. 1A is a schematic illustration of a system according to an embodiment of the invention;

FIG. 1B is a schematic illustration of a system according to further embodiments of the invention;

FIG. 2 is a flow diagram illustrating steps of a method according to an embodiment of the invention; and

FIG. 3 is a schematic illustration of a system operative according to an embodiment of the invention.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well known features may be omitted or simplified in order not to obscure the present invention.

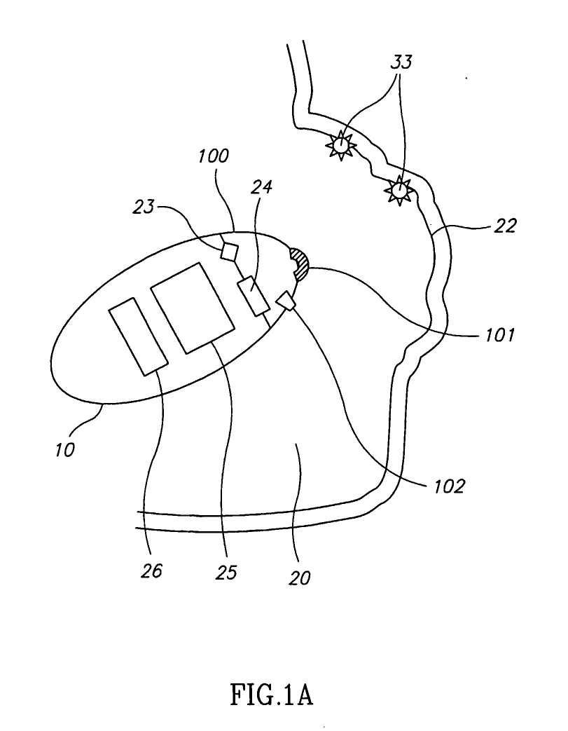

Reference is now made to FIG. 1A, which schematically illustrates a system according to an embodiment of the invention. An in vivo device 10 is capable of detecting products 33 of the bacteria urease reaction in the stomach 20. If H. pylori is present in the stomach 20, urea, which is typically prevalent in the stomach, will be broken down by the bacteria urease. Alternatively, a patient may ingest urea and when the urea reaches the stomach 20 some of it reaches the stomach mucus 22 where, if H. pylori is present, the ingested urea will be broken down by the bacteria urease. The enzymatic reaction is represented as follows:

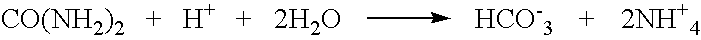

According to one embodiment the product 33 of the enzymatic reaction manifests itself in local pH changes and it is this parameter (pH) that is detected by the in vivo device 10. In alternate embodiments other products (such as ammonia or bicarbonate molecules) may be detected.

In vivo device 10 may be an in vivo pH meter, such as similar to the Heidelberg is capsule or an ISFET pH meter that transmits radio wave signals of endo-luminal pH to an external receiving unit. In alternate embodiments the in vivo device 10 includes an imaging system. For example, device 10 may be an autonomous self contained ingestible imaging capsule. The capsule may be constructed similarly to capsules described in U.S. Pat. No. 5,604,531 and/or in WO 01/65995, both assigned to the common assignee of the present invention and hereby incorporated by reference. An imaging system may include an illumination unit 23, typically comprising a plurality of illumination sources such as white LEDs, an image sensor 24, such as a CMOS or CCD, an optical system (not shown) for focusing an image onto the image sensor, a transmitter 26 for transmitting, such as by RF, image signals of the image sensor 24, and a power source 25, such as a silver oxide battery, that provides power to electrical elements of the imaging device. The device 10 may transmit image and possibly other data to components located outside the patient's body, which receive and process the data. According to one embodiment the illumination unit 23 and image sensor 24 are both disposed behind an optical window 100. pH-sensitive color-changing material 101, such as pH sensitive liquid crystal material (for example, crystal violet lactone) or litmus paper may be placed on the optical window 100. The attachment or placement of the material 101 can be accomplished in several ways. For example, the material 101 may be in the form of a paint, and may be painted onto the capsule. In another embodiment, the material 101 is attached onto the capsule with adhesive. In a further embodiment, the material 101 may be sprayed onto the capsule as a coating. Other attaching methods are possible. Light from the illumination unit 23 is directed towards the pH-sensitive color-changing material 101. The illumination from the illumination unit 23 may be also used to illuminate the body lumen outside of the device 10, such as the stomach 20. In another embodiment a separate illumination source may be included for that purpose. As environmental pH changes cause the pH-sensitive color-changing material 101 to change color, the image sensor 24 may detect the color at each given point in time. Transmitter 26 may transmit the color information to an external receiving unit. In an alternative embodiment a separate transmitter (not shown) may transmit pH data to an external receiving unit. It should be apparent that the color-changing material 101 is in the view of the image sensor 24. Thus, according to embodiments of the invention when a device, such as the described capsule, is swallowed or inserted into the gastrointestinal tract, it may proceed passively through the GI tract while images of the gastrointestinal tract wall and environment may be obtained simultaneously with images of the color-changing material 101. Any color or optical change, due to prevailing pH in the GI environment, will be visible in the images collected from the GI tract, according to one embodiment, providing the viewer with a pH map superimposed on the image.

According to one embodiment a plurality of pH sensitive materials may be used whereas at least one material is sensitive to an acidic pH range (e.g., pH=less than 4.0-3.0) and another material is sensitive to a more basic pH range (e.g., pH=above 5.5). Thus, in a typically acidic environment (e.g., the stomach) basic pH spots (e.g., patches of bacteria) may show up as contrasting colors, thereby facilitating detection.

In an alternative embodiment the device 10 includes at least one sampling chamber 102. The sampling chamber 102 is typically positioned in the field of illumination and in the field of view of the image sensor. According to one embodiment the sampling chamber 102 may be integrated into the device shell, optionally in the optical window 100. Sample chamber 102, according to some embodiments, comprises a chamber cavity enclosed by two sides, a bottom and a membrane, the membrane typically constituting a partition between the body lumen environment and the chamber cavity. According to some embodiments at least the bottom of the chamber 102 may be transparent in the wavelength of illumination. According to other embodiments one or two of the sides are transparent in the illumination wavelength. In alternate embodiments chamber 102 may comprise other components and have other shapes, such as a sack-like or cylindrical shape. Sample chamber 102, which is typically configured for containing endo-luminal samples, such as body lumen fluids, may contain pH sensitive color changing particles, such that color may be apparent in the sample chamber dependant on the sample pH. pH color indicators that may be utilized in this invention are typically weak acids whose colors differ in their dissociated (ionized) and neutral states, having pKa values of from about 6.5 to about 8.5. Typically, the indicators should change color over a pH range of from about 5.5 to about 9.0, preferably from over about 6.5. Examples of appropriate pH indicators may be bromothymol blue, phenol red, p-nitrophenol, neutral red, quinoline blue (cyanine), cresol red and thymol blue. An endo-luminal sample may passively enter the chamber 102 through the membrane. Alternatively, the sample may be actively drawn into the chamber 102, for example, based on osmotic pump technology, wherein flux of fluids into the chamber is typically a function of the membrane pore size and the outside to inside concentration gradient. Alternatively, the sampling can be periodic, controlled, for example, by a switch. Local flux of the stomach environment and displacement of one sample from the sample chamber in favor of another sample may be effected, for example, by using a micro-pump.

The membrane of the chamber 102 may be fabricated from any suitable material, for example from silicon materials and may have any desired cut off size. According to one embodiment the membrane may be semi-permeable with a low molecular weight cut off, e.g. molecular weight of above about 100. According to another embodiment the membrane may be a microscopic mesh with pores in the order of microns. According to some embodiments pH sensitive particles may be immobilized in the chamber 102, such as by being immobilized to a chamber side or bottom or to an appendage that is restricted to the chamber. Chamber 102 may be illuminated by illumination unit 23 such that optical changes, typically as a result of pH or changes in the pH, which may occur in the sample contained in the chamber 102, may be detected by image sensor 24. It will be appreciated that chamber 102 may be made of any suitable material such as plastic, glass etc. Parameters to be considered while assessing if a material is suitable may be, for example, the material's transparency, its safety for internal use, its durability under endo-luminal conditions and so on. Other sampling methods may be used.

According to an embodiment of the invention device 10 is introduced into a body lumen, such as the stomach, and is preferably made to contact the lumen wall. The lumen environment in the vicinity of the lumen wall is then sensed or sampled and analyzed. It has recently been discovered that H. pylori within the stomach is not continuous or in large areas, but rather patchy within the stomach wall. Having the device 10 contact the stomach mucus in a continuous track around the stomach wall or in a few spots along that track, may increase the probability that at least one area of H. pylori bacteria will be sampled or sensed.

According to one embodiment the device 10 is an autonomous wireless device. The device may be capsule shaped or any other suitable shape. For example, the device 10 may be ball shaped to enable rolling the device over the stomach wall with maximal contact points with the stomach mucus. According to other embodiments the device may be tethered, for example, tied to a string that may extend to the patient's mouth to enable holding the device from outside the patient's body to ensure the correct location of the device in the upper GI tract. According to yet other embodiments the device 10 may further comprise one or more appendages attached to the device housing to position and delay the device in the stomach. Alternatively, oil may be administered to a patient to lengthen the device's stay in the patient's stomach. In another embodiment the device may include a magnetic element that can be moved by an external magnetic field, thereby enabling to externally control the device and move it to desired parts of the stomach.

Reference is now made to FIG. 1B which schematically illustrates a system according to an embodiment of the invention. According to one embodiment, located outside the patient's body, are a receiving unit 1000, preferably including an antenna or antenna array 1002, for receiving image and possibly other data from an in vivo device, such as device 10, a receiver storage unit 1004, a data processor, such as processing unit 1006 and a display 1008, such as an image monitor, for displaying, inter alia, the images transmitted by the device 10 and recorded by the receiving unit 1000. Typically, components of the receiving unit 1000 may be small and portable, and may be worn on a patient's body during receiving of in vivo data, such as during recording of the images. For example, antenna array 1002 may include one or more antennas that can be attached to a patient's stomach area, typically positioned to best receive data transmitted from the patient's stomach. The antenna array 1002, may be part of a belt or garment worn about the relevant portion of a patient's body.

Preferably, the processing unit 1006 and display 1008 may be part of a personal computer or workstation, which includes standard components such as a processor, a memory, a disk drive, and input-output devices, although alternate configurations are possible. A receiving system similar to embodiments described in the above mentioned U.S. Pat. No. 5,604,531 and/or in WO 01/65995 may be used.

According to one embodiment the processing unit 1006 is capable of being fed pH data and is capable of outputting indication of a pH or of a pH change which exceeds a predetermined threshold. For example, pH data from a patient's stomach can be transmitted to the receiving unit as described above, for example, as electronic signals that can be translated into pH values or as part of image data that is being transmitted from an in vivo imaging system. According to one embodiment the processing unit 1006 is capable of indicating a pH value that is equal to or larger than about 5.5. According to other embodiments the processing unit 1006 is capable of calculating a pH change and indicating when this change exceeds a threshold. For example, a threshold call be determined as the difference between a normal stomach pH (which is about 2.0-3.0) and a more basic area (e.g., 5.5 ), which typically indicates the presence of H. pylori, according to embodiments of the invention. Thus, a threshold of over about 2.5 may be indicated as a sign for the presence of the bacteria. According to some embodiments a threshold may be determined according to calibration procedures.

Typically, indication of a pH value or pH change, according to embodiments of the invention, is relayed to display 1008. The display 1008 may include any suitable display means such as an image monitor, a graphic display, a light indicator an audio display, a vibrating mechanism, and so on.

It should be appreciated that components of the receiving unit 1000 may be part of one unit or work station or may be separate units which are in electric communication, for example, by a wired or wireless connection.

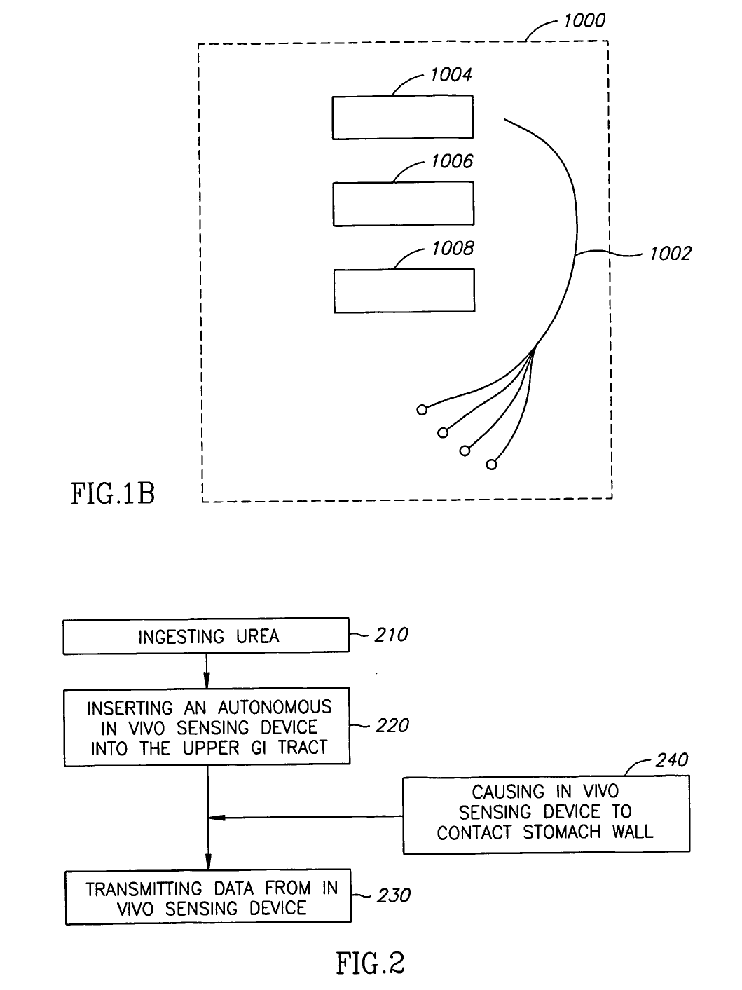

Reference is now made to FIG. 2, which illustrates steps of a method according to an embodiment of the invention. In a first step (210 ) a patient ingests urea, preferably in liquid form, for example, in a volume of 50 ml. The urea reaches the stomach and may come in contact with the stomach mucus. According to one embodiment urea is suspended in oil, such that it is delayed in the stomach. According to other embodiments urea can be added to heavy beads that will bring the urea to the stomach wall. In alternative embodiments it is not necessary to ingest urea. Normally prevailing urea may be used by the bacteria to produce bases.

In a second step (220) an in vivo device—such as an imaging device having intrinsic pH sensing capabilities (for example, as described above)—is inserted into the patient's upper GI tract, for example into the stomach. The device may be inserted by ingesting the device, by placing it, for example with the aid of an endoscope, and so on. Typically, there may be an interval (e.g. a 20 to 30 minute interval) between the step of ingesting urea and the step of inserting the in vivo sensing device.

In a third step (230) in vivo data is transmitted from the device. According to one embodiment pH data is transmitted to an external receiving unit According to another embodiment images of the stomach are obtained and transmitted to an external operator either on line or off line (for example, images are transmitted to a recorder which is reviewed only later). According to some embodiments, images obtained by devices such as described above, also include pH data (for example, color indication of the prevailing pH in areas of the stomach). Following the ingestion of urea the prevailing pH may be an indicator of the presence of H. pylori, since relatively basic areas are expected due to H. pylori enzymatic activity. Thus, by utilizing a method according to some embodiments of the invention, the prevailing pH can be visualized and therefore also the presence of H. pylori can be visualized.

An additional step (240), according to some embodiments of the invention, includes causing the in vivo device to contact the stomach wall (preferably, the stomach mucus) in at least one spot. According to one embodiment this step may include mixing and/or increasing pressure in the stomach lumen. This may cause an autonomous in vivo sensing device that is situated in the stomach to be pushed to the stomach wall and to contact it. According to one embodiment a patient may intake a carbonated beverage prior to inserting an in vivo sensing device. The carbonated beverage typically causes an increased pressure in the stomach. Furthermore, a carbonated beverage, or any other suitable solution may induce belching, which typically causes movement and turbulence of the stomach content in addition to causing increased pressure, so as to move an autonomous in vivo sensing device from one location to another along the stomach wall.

According to another embodiment a patient may be specifically positioned so as to cause an in vivo sensing device, such as a capsule as described above, which is situated in the stomach, to move along a predictable path to a particular region of the stomach.

The following examples disclose possible procedures according to embodiments of the invention. Other amounts and times may be practiced according to other embodiments of the invention.

### EXAMPLES

Procedure 1 for Contacting an Autonomous pH Sensing Capsule with the Stomach Mucus:

1. Patient swallows urea in 50 ml of water;

2. immediately after ingestion of urea 8 ounces of carbonated water are administered to the patient;

3. patient waits 20 to 30 minutes and then swallows an M2A™ capsule having pH sensitive liquid crystal material glued to the optical window.

Procedure 2 for Positioning a Patient so as to Ensure Contact of an Autonomous pH Sensing Device with Predetermined Spots on the Stomach Wall:

1. While lying flat on his left side, the patient swallows a pH sensing capsule with 50 ml of water;

2. The patient is positioned Back Trendelenburg −10 degrees for about 1 minute;

3. The patient is positioned on his left side with his head elevated at 45 degrees for about 1 minute;

4. The patient is positioned on his left side with his head elevated at 60 degrees for about 1 minute;

5. The patient is positioned on his left side with his head elevated at 80 degrees for about 1 minute;

6. The patient is positioned flat on his back, then flat on his left, flat back again, flat on his abdomen and then flat on his right side for about 1 minute.

Note: a step of ingesting urea may precede these positioning steps.

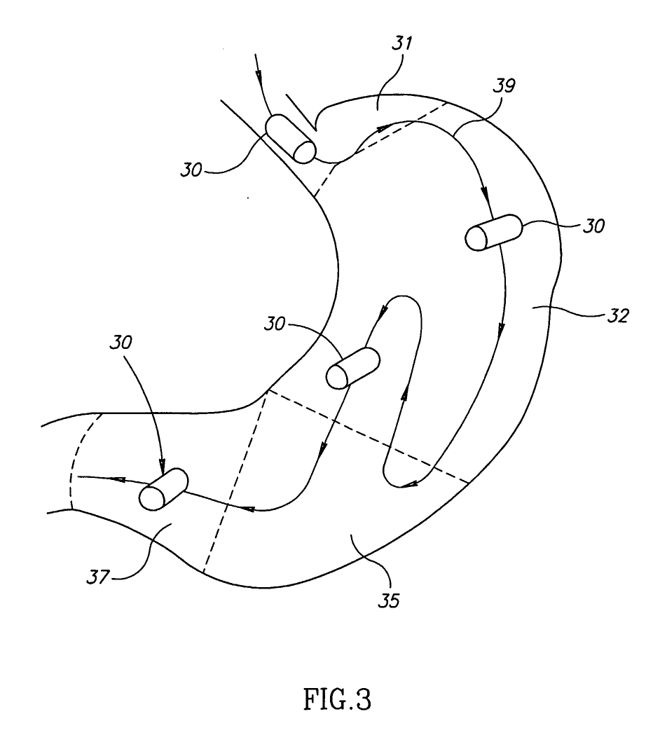

FIG. 3 schematically illustrates a swallowable capsule having pH sensing capabilities that is moved about a patient's stomach according to an embodiment of the invention, for example, according to the above Procedure 2. It can be seen that the capsule follows a track that covers most of the stomach body, including remote areas such as the cardia, fundum, antrum and the pyloric canal. It will be appreciated that rotating the patient causes the capsule to be moved by its own weight and to rest directly on the stomach wall (mucus) at each position. It will be appreciated by persons skilled in the art that the present invention is not limited by what has been particularly shown and described herein above. Rather the scope of the invention is defined by the claims, which follow.

1. A system for in vivo detection of H. pylori, the system comprising:
an autonomous in vivo sensing device configured for sensing in vivo pH and for transmitting in vivo data to a receiving unit, said sensing device comprising an imager; and
an external receiving unit configured for indicating an in vivo pH, based on the transmitted in-vivo data.

2. (canceled)

3. The system according to claim 1 wherein the external receiving unit is configured for indicating an in vivo pH about equal or larger than 5.5.

4. (canceled)

5. The system according to claim 1 wherein the sensing device includes pH indicator.

6. The system according to claim 5 wherein the pH indicator is a color changing indicator.

7. (cancelled)

8. The system according to claim 6 wherein the pH indicator is attached to an optical window in the sensing device.

9. The system according to claim 6 wherein the pH indicator is immobilized within a sampling chamber in the sensing device.

10. (canceled)

11. The system according to claim 1 wherein the sensing device comprises a radio frequency transmitter.

12. The system according to claim 1 wherein the sensing device comprises a power source.

13. (canceled)

14. The system according to claim 1 wherein the receiving unit is configured for receiving radio frequency signals.

15. The system according to claim 1 wherein the receiving unit comprises a display configured for displaying transmitted in vivo data.

16. (canceled)

17. A system for in vivo detection of H. pylori, the system comprising:
an autonomous in vivo pH sensing device, said device comprising an imaging system and a transmitter;
an external receiving unit; and
a processor configured for identifying changes in pH over a predetermined threshold.

18. The system according to claim 17 wherein the predetermined threshold includes a pH change of about 2.5 units.

19. The system according to claim 17 further comprising a display.

20. (canceled)

21. (canceled)

22. A method for in vivo detection of H. pylori, the method comprising
sensing pH in at least one location proximate to a patient's stomach mucus; and
transmitting by radio frequency pH data to an external receiving unit.

23. The method according to claim 22 further comprising indicating a pH value which is about equal to or exceeds a predetermined threshold.

24. The method according to claim 22 wherein sensing pH is by imaging a color changing pH indicator.

25. The method according to claim 23 wherein the pH value is about 5.5.

26. (canceled)

27. The method according to claim 23, the method comprising:
inserting an autonomous pH sensing device into a patient's stomach;
positioning the patient to achieve substantially covering of the patient's stomach body; and
receiving in vivo data.

28-29. (canceled)
